(19) United States
(12) Patent Application Publication
(10) Pub. No.: US 2011/0144495 A1
(43) Pub. Date: Jun. 16, 2011

(54) Perfusion Imaging of a Volume in Medical Diagnostic Ultrasound

(75) Inventors: Wilko Wilkening, Mountain View, CA (US); Saurabh Datta, Cupertino, CA (US)

(73) Assignee: Siemens Medical Solutions USA, Inc., Malvern, PA (US)

(21) Appl. No.: 12/637,493

(22) Filed: Dec. 14, 2009

(51) Int. Cl. A61B 8/14

(52) U.S. Cl. 600/443

(57) ABSTRACT

A volume is scanned with ultrasound for determining perfusion. A volume is scanned with a more rapid technique for tracking a sub-volume, and the tracked sub-volume is scanned for contrast agent detection with a less rapid technique. For example, a single pulse technique or B-mode scanning is used to track a region over one or more cycles, the location of the tracked region is predicted, and multiple pulse contrast agent detection is performed for the sub-volume at the predicted location. The combinations of scanning provide for real-time or higher temporal resolution reperfusion information at the appropriate tissue. Using a separate scan for motion tracking may provide a more robust prediction of the sub-volume location and a better visualization of the results (e.g., orientation within the organ). In other embodiments, tracking is based on a B mode image derived from the multi-pulse data.

### BACKGROUND

The present invention relates to perfusion imaging with ultrasound. In particular, medical diagnostic ultrasound is used to detect contrast agents to determine perfusion in moving tissue.

Contrast agents are injected in the blood stream to monitor the flow of blood into organs, such as the myocardium. Contrast agents typically take several heart beats to completely penetrate the entire myocardial muscle. Changes in the regional perfusion can be an indicator of ischemia, and mapping the changes can help clinicians make correct diagnosis. The perfusion in a region of myocardium can be evaluated by a destruction-reperfusion sequence.

The contrast agents in the blood volume present in the muscle appear less bright due to smaller blood volume in the muscle as compared to the adjacent cavity. Certain pulse sequences are employed to enhance signals from the contrast agent and suppress signal due to the muscle, improving sensitivity and specificity. This approach also improves the signal-to-noise ratio in the imaging. The use of the contrast specific pulse sequence requires several transmit/receive cycles per scan line.

Destruction of contrast agents and detection of reperfusion is conventionally done in two-dimensional slices. However, reperfusion can occur from anywhere outside the slice. The contrast agents exist in a larger volume around the slice. The map of perfusion for a two-dimensional slice may not account for motion of the tissue outside of the scan plane, repositioning of the scan plane, and concentration differences around the scan plane.

For scanning a volume or plane, the contrast specific pulse sequence is limited to a lower temporal resolution due to the multiple pulses. Temporal resolution is a significant challenge in acquisition of data for a full volume, compounded by a multiple pulse technique. Temporal resolution is important due to the rapid motion of the heart muscle and to properly detect reperfusion. Movement of the heart can result in inhomogeneous destruction of contrast agents. Reperfusion may come from compartments where microbubbles were partly destroyed and time intensity curves may represent different locations over time rather than the same location.

### BRIEF SUMMARY

By way of introduction, the preferred embodiments described below include methods, instructions, and systems for perfusion imaging of a volume in medical diagnostic ultrasound. A volume is scanned with a more rapid technique for tracking a sub-volume, and the tracked sub-volume is scanned for contrast agent detection with a less rapid technique. For example, a single pulse technique or B-mode scanning is used to track a region over one or more cycles, the location of the tracked region is predicted, and multiple pulse contrast agent detection is performed for the sub-volume at the predicted location. The combinations of scanning types provide for real-time or higher temporal resolution reperfusion information at the appropriate tissue. Using a separate scan for motion tracking may provide a more robust prediction of the sub-volume location and a better visualization of the results (e.g., orientation within the organ). In other embodiments, tracking is based on a B mode image derived from the multi-pulse data.

In a first aspect, a method is provided for perfusion imaging of a volume in medical diagnostic ultrasound. A volume of a patient is first scanned with single pulses for each transmit scan line. First ultrasound data is received in response to the single pulses. The first scanning and receiving are performed a plurality of times during a first physiological cycle. A region of interest of the patient within the volume is determined from the first ultrasound data of at least one of the first scans. The region of interest is tracked between the repeated first scans with the first ultrasound data from the repeated first scans. A first next location of the region of interest is predicted as a function of the tracking. A sub-volume of the volume is second scanned. The sub-volume corresponds to the region of interest at the first next location. Beamforming parameters are set for the first next location to perform the second scanning of the sub-volume and not laterally outside the sub-volume. The second scanning is with acoustic energy for destroying contrast agents. A second next location of the region of interest is predicted as a function of the tracking. The sub-volume is third scanned with the beamforming parameters set for the second next location. The third scanning is with acoustic energy for detecting the contrast agents. The contrast agents are detected in response to multiple pulses from the third scanning. The third scanning and detecting are repeated a plurality of times during a second physiological cycle. An image representing perfusion over time of the contrast agents in the sub-volume is generated. The image is responsive to the detected contrast agents of the repetitions of the third scanning.

In a second aspect, a system for perfusion imaging of a volume in medical diagnostic ultrasound is provided. A transducer and beamformer are configured to scan a volume and a sub-volume. The volume is larger than the sub-volume, and a first scan of the sub-volume is configured to destroy contrast agents in the sub-volume. A detector is configured to detect response to the scan of the volume. A processor is configured to track a moving region of tissue as a function of time within the volume using the response to the scan of the volume. The beamformer is configured, as a function of the tracking, to perform second scans of the sub-volume at different locations at different times such that the sub-volume has a defined spatial relationship with the moving region of tissue, such as including the region. A contrast agent detector is configured to detect contrast agents in response to the second scans of the sub-volume. A display is configured to display an image, where the image is a function of the detected contrast agents.

In a third aspect, a computer readable storage medium has stored therein data representing instructions executable by a programmed processor for perfusion imaging of a volume in medical diagnostic ultrasound. The storage medium includes instructions for tracking tissue motion in three-dimensions of a first volume, adjusting, as a function of the tracking, beam steering to account for the tissue motion, the adjusting being ongoing throughout a physiological cycle, transmitting acoustic energy destructive of contrast agents with the adjusted beam steering, and detecting, after the transmitting of the acoustic energy destructive of the contrasts agents, reperfusion in a second volume over time, the detecting performed with the adjusted beam steering, the second volume being a same volume as the first volume or being based on the tracking of the first volume.

The present invention is defined by the following claims, and nothing in this section should be taken as a limitation on those claims. Further aspects and advantages of the invention are discussed below in conjunction with the preferred embodiments.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flowchart diagram of one embodiment of a method for perfusion imaging of a volume in medical diagnostic ultrasound;

FIG. 2 is an example graphical representation showing various regions in a cross-section of a scan volume;

FIG. 3 is an example graphical representation showing motion of a region of interest in a heart;

FIG. 4 is an example graphical representation showing motion, due to heart movement, of a region of tissue relative to a region of interest box;

FIG. 5 is an example graphical representation showing displacement of a region of interest box with a region of tissue moving due to heart motion;

FIG. 6 is an example graphical representation showing displacement of two region of interest boxes with two respective regions of tissue moving due to heart motion; and

FIG. 7 is a block diagram of one embodiment of a system for perfusion imaging of a volume in medical diagnostic ultrasound.

### DETAILED DESCRIPTION OF THE DRAWINGS AND PRESENTLY PREFERRED EMBODIMENTS

Perfusion maps based on the two-dimensional slices do not account for out-of-plane motion of tissue. For creating perfusion maps in three-dimensional and specifically in a fast moving organ, both temporal and spatial registration is important. Three-dimensional perfusion is assessed in real time using three-dimensional or volume ultrasound images. By utilizing specific pulse and imaging sequences designed to enhance contrast and specificity and by using tissue motion tracking for a region/sub-volume of interest, the scanning may be performed more rapidly. The tracking is performed with more rapid scanning or fewer pulses per receive line. Contrast agent scanning is performed with multiple pulse techniques, but for a sub-volume. Interleaving the two types of scanning allows for more rapid overall scanning as compared to using multiple pulses for contrast agent detection throughout the entire volume. High volume rate imaging with contrast specific acquisition establishes and maintains the spatial-temporal correspondence between the volume of interest and a reperfusion location with respect to the anatomy. Once the spatial matching is achieved, the temporal persistence for that region can be used to quantify perfusion.

Perfusion is assessed where the reperfusion process within a given tissue structure is followed over time. Real-time three-dimensional imaging allows for tracking and detection of reperfusion. A volume is tracked in three-dimensions over time. Tracking may include tracking of regions in the tissues as well as surfaces, such as the surface of the left ventricle. Single pulse scanning of a larger volume is performed for the tracking of the contrast agent volume. Out-of-plane motion contributes to inaccuracies in two-dimensional approaches, but three-dimensional tracking avoids or limits these inaccuracies.

Beam steering, tracking, and motion prediction allow destruction of microbubbles in a well defined smaller volume. This smaller volume is tracked. Beam steering for contrast agent detection is adjusted according to motion predications from the tracking or in combination with knowledge-based motion prediction algorithms. The destruction and subsequent reperfusion detection, image processing, and/or quantification are performed for the smaller volume at the appropriate tissue locations and resolution (e.g., higher resolution for reperfusion detection than for tracking). Contrast agent beamformation and detection are provided for the predicted location of the smaller volume and not other lateral locations despite tissue motion along any direction. Regional perfusion study allows reconstruction of a three-dimensional perfusion profile for an entire or portion of an organ with geometric consistency. Exam protocols for ischemic cardiomyopathy, coronary artery disease, or other conditions may benefit from the three-dimensional reperfusion study. Software tools and user interface capabilities may allow more accurate perfusion quantification.

The heart and heart cycle are used as an example. Other uses are possible, such as the liver where breathing introduces a fair amount of motion.

Using the tracking and perfusion scanning, some region specific image processing can be performed and/or regional computation or quantification of contrast specific parameters can be made. The image processing or quantification can be performed for one or more heart cycles.

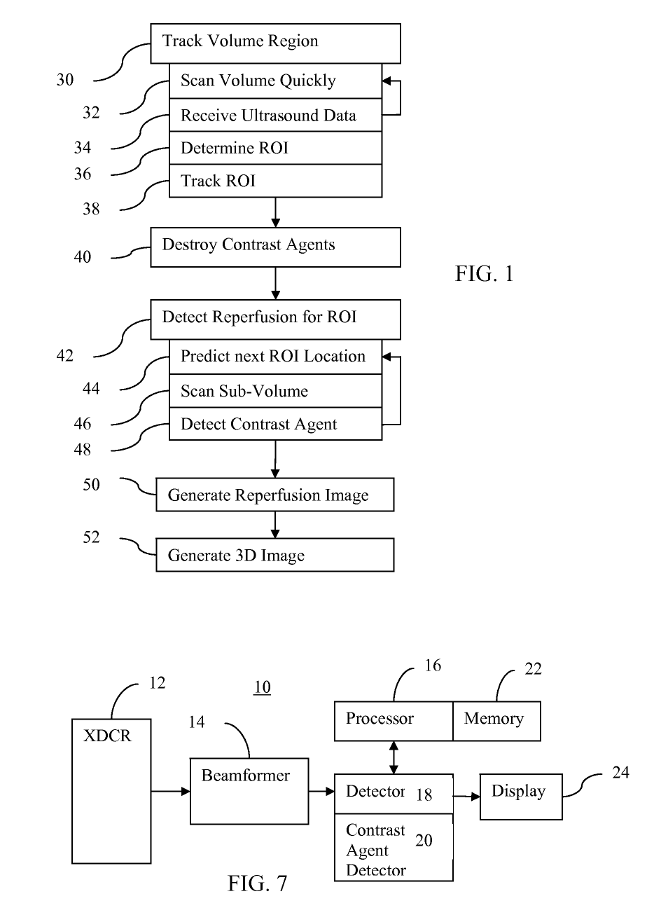

FIG. 1 shows a method for perfusion imaging of a volume in medical diagnostic ultrasound. The method is implemented on the system 10 of FIG. 7 or a different system. Additional, different or fewer acts may be provided. For example, acts 30 and 42 are performed without acts 40, 50, and/or 52. Acts 30 and 42 are associated with more specific acts 32-38 and 44-48, but may be performed without these specific examples, with additional specific acts, with fewer specific acts, and/or with different specific acts. The acts are performed in the order shown, but may be provided in other orders.

In act 30, a history of tissue motion is determined. The tissue is tracked in three-dimensions with or without rotational, scale and/or deformation tracking. The tracking is performed for a volume. For example, B-mode scanning and detection are performed for a volume larger than a tissue region of interest. The volume is sufficiently large to allow the motion of the tissue without the tissue moving substantially (e.g., sufficiently to jeopardize the tracking) outside of the statically positioned volume. The volume scanned for tracking is larger than the sub-volume or volume for which reperfusion is to be studied. Tracking a larger volume may be more robust due to a greater number of anatomy features being available for tracking. The larger volume may allow for tracking multiple sub-volumes with the same scans.

Acts 32-38 provide an example embodiment for tracking, performed in the order shown or a different order (e.g., act 36 being performed before acts 32 and 34). In act 32, the volume is scanned. The volume is scanned with electronic, mechanical, or both electronic and mechanical steering. A plurality of sequential transmit and receive events are performed to scan the volume with ultrasound. In one example, broad transmit beams are formed for receiving respective pluralities of receive beams (e.g., receive sixteen or more receive beams in parallel in response to each transmit beam). Any scan format may be used, such as linear, sector, or Vector®.

To scan the volume rapidly (i.e., higher temporal resolution), a single pulse technique is used. For example, B-mode detection determines the intensity of the echoes for a given location in response to a single transmit beam. Data along each receive scan line is detected from only one transmit pulse for a given frame. A pulse of acoustic energy is generated from one or more elements of a transducer. Each element generates acoustic energy for the pulse in response to electrical waveforms. Each electrical waveform may include one or more cycles, such as 1.5 cycles. Multiple transmit pulses are generated for scanning different locations in the volume. Other single pulse techniques may be used. In other embodiments, multiple pulse detection may be used, such as receiving along a given scan line multiple times in response to multiple transmit pulses. Multiple pulse detection includes Doppler detection. Combinations may be provided, such as scanning one portion of the volume with single pulses and another portion with multiple pulses per receive scan line.

The temporal resolution may be increased by using fewer receive and/or transmit scan lines with or without sparse sampling. Low spatial resolution allows for fewer transmit and respective receive events to scan the entire volume. Lowering the spatial resolution increases the frame rate. Other approaches, such as transmission with larger or more spread out wave fronts and more parallel receive beamformation, may be used to increase the frame rate for the volume scan.

The ultrasound data is acquired over the entire or other spatial extent with a same or different scan line density than for subsequent reperfusion or destruction scanning. Data representing at least portions of the three-dimensional volume are acquired for positions at least partially around the region of interest. A lesser line density, sample density or combinations thereof may be used. In one embodiment, the ultrasound data is equally or evenly spaced throughout the volume, but unequal or variations in sample or line density may be provided. In one embodiment, the data for the entire volume is acquired with a low resolution, such as using a low frequency or smaller aperture. Low resolution may result in a higher frame rate for scanning the entire spatial extent.

In act 34, ultrasound data is received in response to the transmit pulses. Acoustic echoes reflect back to the transducer array or elements. The elements convert the acoustic echoes into electrical energy. The received ultrasound data is the channel data output for each element, beamformed data, or detected data. For example, the ultrasound data is beamformed data representing one or more (e.g., 16) receive scan lines. The ultrasound data is formed from analog information or digital samples.

The scanning and receiving of acts 32 and 34 are repeated sequentially to scan the volume. Alternatively, a single transmit or broad beam transmit may be used. A frame of data representing the entire volume is acquired.

The volume scan (scanning and receiving of acts 32 and 34) is performed multiple times. For example, the volume is scanned a plurality of times during a portion of a heart cycle. As another example, the volume is scanned a plurality of times during one or more heart cycles. Any frame rate may be provided, such as scanning 20, 30, or more times in a second or in a heart cycle. Scanning and receiving may be performed for other physiological cycles, such as the breathing cycle.

In one embodiment, the scanning and receiving are interleaved with scanning for destruction of contrast agents in act 40 and/or scanning for detecting reperfusion in act 42. Any interleaving may be performed, such as scanning the volume partially, one time, or a plurality of times for each of the tracking scan and the detection scans.

In act 36, a region of interest of the patient within the volume is determined. The region of interest corresponds to the tissue for which the reperfusion is to be studied, such as a region surrounding or including the myocardium. The region of interest may be a cube, sector cone, Vector® cone, or other shape. At least a portion of the tissue for reperfusion study is within the region of interest. The region of interest may be larger than the portion of the tissue to be studied. The sub-volume of the region of interest is large enough so that some structures within the sub-volume may not move out of the sub-volume during the reperfusion process. Alternatively, the sub-volume is smaller or is based on detection of the specific tissue border.

The region of interest is identified within the scan volume. The received ultrasound data may be automatically processed to locate features, such as boundaries associated with heart muscle. The region for perfusion quantification may be selected by user in three-dimensions from images generated from the received ultrasound data. An image may be generated and the user may indicate the region of interest, such as positioning a three-dimensional cube or selecting regions from orthogonal two-dimensional images. As an alternative to automatic or manual determination, semi-automated approaches may be used. For example, the user selects one or more anatomy features from an image. A processor determines the tissue of interest based on the features and a bounding shape enclosing the tissue.

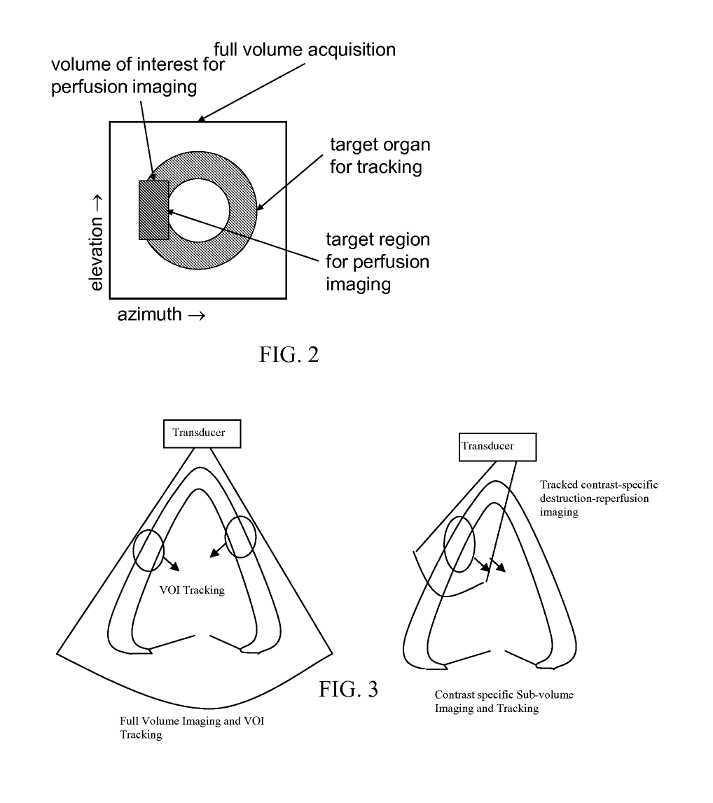

FIG. 2 shows a plane of constant depth or range (C plane) from a three-dimensional volume acquisition of the target organ. The C plane is used for simplicity. The actual regions are three-dimensional, so determination of the sub-volume occurs in three-dimensions. The target organ is represented as a circular shape, such as a cross section of the heart. A portion of the target organ is selected for reperfusion study, such as represented by the darker target region. A volume or sub-volume surrounding the tissue is determined and represented as a box. The sub-volume is to be used for tracking and also serves as the target region for perfusion imaging.

The ultrasound data used for determining the region of interest is a frame of reference used for tracking. The region of interest is indicated relative to a set of ultrasound data representing a particular scan or given time. The reference data is used for tracking.

In act 38, the region of interest is tracked. The displacement, rotation, scale, deformation or combinations thereof of the tissue of interest or the region of interest is tracked. The tracking is between different scans. Each scan represents the patient at a different time. The tissue moves between, and in part during, each scan. The region of interest is tracked to determine the amount of movement or the location of the tissue at different times.

The information for the tracking is gathered before the destruction/reperfusion sequence is started. The tracking is used to develop a cyclical history or current trend of movement of the tissue. The same tissue likely moves to the same locations at the same times during each cycle. The region of interest may be repositioned and resized over time to optimally cover the structure of interest. The tracking information is determined for one or more beats to estimate of motion of the volume of interest with respect to the anatomy. Alternatively, the tracking information over a portion of a cycle indicates a trend, reflecting likely direction and amount of continued movement. In other embodiments, tracking is performed interleaved with scanning for contrast agents without an initial history of motion. The trend is used to predict subsequent locations.

FIG. 3 shows tracking a region of interest represented by a circle or oval. For example, a high frame rate full volume image acquisition with single pulses for each line is used for tracking the region of interest throughout the motion cycle. The arrow indicates movement of the tissue, such as heart wall, towards a center of the scan volume. The tracking allows scan of a smaller volume directed to the expected location of the tissue based on past or history of tissue motion. Contrast specific destruction and reperfusion image acquisition sequences are performed at these predicted locations.

The region is tracked from frame to frame using any now known or later developed tracking algorithm. Using speckle tracking or tracking of features (e.g., applying gradient processing and then tracking peak gradient locations), any motion of the tissue relative to the reference is determined. For example, any of various constant or adaptive search processes are used to provide a best match or a sufficient match of each of the sub-volume or region of interest to the reference frame of data. Translation and/or rotation along three dimensions are determined. The resulting translational and rotational vectors are combined, such as through averaging, to identify an overall motion. A single vector in three-dimensions may be determined.

The reference frame may be updated, such as using a most recent frame for which the position of the tissue is known as the reference frame. Alternatively, the frame used for the initial determination of the region of interest is used for tracking throughout the cycle. Acquisition parameters for obtaining ultrasound data for motion tracking are the same or different than used for acquiring the reference information. In an alternative embodiment, acquisition of the tracking data is adaptive. For example, the size of each beam, the number of beams or other acquisition parameter is adjusted as a function of a previous motion estimate, the variance associated with the motion estimate, or a measure of tissue rigidity. For large variance motion estimates or low tissue rigidity, the beam size is increased or the number of beams is increased. The acquisition parameters may also be updated as a function of a change in acquisition parameters for imaging. For example, the user selects a different center frequency, aperture, F number, depths of imaging or other imaging parameter. The same parameter is altered for obtaining tracking data.

A motion vector is determined for the sub-volume. The sub-volume is tracked as a whole. Alternatively, the sub-volume is divided into smaller regions, each region is separately tracked, and the motion vectors for the smaller regions are combined, such as averaging, or used to determine deformation. The motion vector provides a direction, a magnitude or both a direction and a magnitude of the motion.

Motion is detected by comparing data acquired at different times, such as comparing each subsequently acquired set of data with the reference frame of data. In one embodiment, motion vectors are determined by tracking using speckle correlation. A high pass filter or other filtering and acquisition parameters are selected to best identify or provide speckle information. In alternative embodiments, a spatial gradient is applied to the data to identify one or more features within the region of interest. Easily identified landmarks, such a cystic areas, blood vessels or highly echogenic specular targets are tracked instead of tracking pixels within a sub-volume for speckle correlation.

A minimum sum of absolute differences, cross correlation, or other now known or latter developed correlation is used to match the data for a scan with the reference data. Correlation is performed using data prior to detection, data after detection but prior to scan conversion, data after scan conversion, display image data, or other data. Any of various search patterns involving translating, deforming, scaling, and/or rotating the data for one scan relative to the reference data is used to identify a best match. A coarse search followed by a fine search, a search adapted to expected motion, a size of the region to be searched adapted to previous amounts of motion, or other adaptive or efficient search techniques may be used.

In one embodiment, a warping, such as one-, two- or three-dimensional expansion or contraction of the data is performed as part of the correlation operation. By spatially expanding or contracting (i.e., scaling) the data, the data more likely matches the reference data. Other warping may be used. Rigid body, non-rigid body, or other types of transformations may be used.

In one embodiment, the sub-volume used for tracking includes (e.g., surrounds) the region of tissue. In other embodiments, the sub-volume has a defined spatial relationship with the moving tissue region, but may not include a any portion or all of the tissue region. The tissue region in itself is not tracked, but is located at a predictable location relative to the separately tracked region.

The tracking may alternatively or additionally include motion modeling. For example, the location prediction is augmented by a predefined motion model. A knowledge-based algorithm models motion of the tissue and is used to predict the locations.

The motion vectors for different scans correspond to different times in the physiological cycle. A history of motion is determined based on the cycle phase or a trend. The motion over multiple cycles may be averaged, such as determining an average motion or location of the tissue, for each phase from multiple cycles.

In act 40, another scan is performed to destroy contrast agents. Acoustic energy destructive of contrast agents is transmitted. The acoustic energy is from a point source or an array. A converging, plane (infinite focus), or a diverging focus may be used. A single or multiple transmissions along a same, adjacent or different scan lines may be used. The acoustic energy has a relatively high mechanical index at an elevation focal point, a steered focal point, or other location. Relatively high is 1.0 or higher, but lower mechanical index values may be used. The acoustic energy is within any required limits, such as being 1.6 or less, but may exceed the limits if allowable. The acoustic energy for destruction is the same, higher, or lower mechanical index as used for the tissue scanning in act 32. In one embodiment, the sequential transmissions for destruction of contrast agents during a perfusion study described in U.S. Pat. No. 6,340,348, the disclosure of which is incorporated herein by reference, are used. For contrast agent destruction, higher amplitude and lower frequency may more likely destroy the contrast agents. Any number of cycles may be used, such as 3-10 cycles.

The acoustic energy for destruction is transmitted to less than the entire volume. For example, the sub-volume is scanned with focal locations in the sub-volume. Regions laterally outside the sub-volume are not subjected to or subjected to lesser acoustic energy, resulting in lesser or no destruction. Regions along the scan lines for destruction but away from the focal locations may be subjected to less acoustic energy as well. The acoustic energy propagates into a region with contrast agents. The region is the sub-volume along the propagation path. The region may be a region of interest, an organ, a portion of an organ, a fluid cavity, a vessel or other location. For example, FIG. 3 shows a scan region for destruction being over the region of interest but not the entire volume.

The transmit beams for destruction are adjusted to scan the region of interest and not other locations. The beam steering is positioned based on the predicted location of the tissue of interest. The spatial extent of the sub-volume of interest is used to define the beam span for the contrast specific pulses as well as destruction-reperfusion sequence.

The location is predicted based on the time within the cycle and the motion determined at that time from the tracking. For example, the destruction is to be performed at time T+1. The region of interest at time T+1 in previous cycles is located at a specific location with a specific size. The beamformer is configured to scan the sub-volume at this predicted location. As another example, the location of the region of interest in the current cycle at time T is determined by tracking. The magnitude, direction, and/or deformation of the region of interest from T to T+1 in previous cycles is added to the location at current cycle time T to predict the location for the current cycle at time T+1. Other approaches for predicting the location of the tissue or region of interest from the tracking may be used, such as extrapolating based on a trend of motion through the current cycle. Combinations of approaches may be used, such as tracking a trend and using cycle history and averaging the resulting predicted vectors.

Destruction of contrast agents may take some time. Multiple firing with different transmit foci may be used to achieve the desired destruction. The location of the destruction scan may be repositioned during destruction using the tracking. Tracking may also be used to avoid more likely erroneous results. For example, the destruction process is halted or ceases if the tissue has moved too much.

For assessing perfusion, the contrast agents in the region of interest are destroyed. The rate of reperfusion from this controlled time may be determined.

In act 42, perfusion is detected. Where contrast agents were destroyed in act 40, reperfusion is detected. After transmission of the destructive acoustic energy, reperfusion of contrast agents is detected. The reperfusion is imaged using any technique, such as a contrast agent mode. In principle, the contrast agent concentration increases more rapidly in regions with high perfusion rates. The perfusion at the region of interest is detected over time. The tracking is used to predict the location of the region over time for detecting the perfusion, allowing contrast agent detection just for the region of interest. The use of tracking may reduce scan time and provide location specific perfusion rate despite organ motion.

Acts 44-48 provide an example embodiment for detecting reperfusion, performed in the order shown or a different order. In act 44, the location, size, and/or shape of the region of interest or tissue location is predicted. A next location of the region of interest is predicted using the tracking information. The next location is predicted from previous motion in a different physiological cycle or a trend. By determining motion of the tissue at a same phase from a different cycle, the motion of the tissue in a later cycle is predicted. Information from an ECG device may additionally be used to determine the exact phase of heart cycle to assist in assignment of cycle phase and improve accuracy. Any of the approaches to prediction discussed above for act 40 may be used. The tracking of act 30 allows prediction of the next location, so geometric consistency is maintained.

In one embodiment, the sub-volume being tracked is the same as the sub-volume used for destruction and for detection of reperfusion. The same prediction approach is used for both destruction and reperfusion detection. Tracking during perfusion is supported by the tracking information derived from the larger volume. In other embodiments, the sub-volumes are different and/or different prediction approaches are used. The sub-volume corresponding to the region of interest may change in size and/or shape based on the tracking or may be larger or smaller for destruction than for perfusion detection.

In act 46, the sub-volume or region of interest is scanned. The sub-volume is scanned by adjusting the beam steering to account for tissue motion. The prediction of act 44 is used to set the scan locations. For example, FIG. 3 shows a subsequent scan for perfusion detection where the scan just covers the region of interest or less than the entire volume. Contrast agent specific scanning is not performed outside the sub-volume.

The scan is for contrast agent detection. Any contrast agent detection scan mode may be used, such as B-mode. In one embodiment, multiple pulse techniques are used. Two or more transmissions along the same or adjacent transmit scan lines are fired. For example, different amplitudes and phases are used on three transmissions to provide for nonlinear fundamental response. As another example, two pulses, such as two opposite phase pulses, are used to isolate response at even harmonics. Contrast agents may have a stronger response than tissue at even harmonics.

The scanning of act 46 is performed with a lesser amplitude, higher frequency of the waveform, and/or less power (e.g., fewer cycles) than the acoustic energy for destruction in act 40. For example, the mechanical index of the scanning in act 46 is 0.6 or less. These characteristics are used to maintain the contrast agents without substantial destruction. Substantial accounts for the destruction of some but not a majority of the contrast agents due to any acoustic energy. By avoiding destruction in the scanning for reperfusion, the reperfusion rate may be determined.

In one embodiment, the scanning for tracking of act 30 is ongoing or regularly performed for more accurate prediction on an ongoing basis. The scanning of act 30 is interleaved with the scanning of act 46. The system alternates between the full volume acquisition for tracking and the contrast specific imaging of a smaller sub-volume for reperfusion detection. The sequence can be altered or adjusted in a way that one or more fast large volume acquisitions are followed by one or more contrast specific sub-volume acquisitions to improve tracking or perfusion estimation considering motion of the organ and perfusion dynamics. Partial scans may be interleaved (e.g., scan half the full volume, scan one or more sub-volumes, scan the other half of the full volume, and repeat). The interleaving may adapt so that more tracking information is acquired during times of rapid tissue movement and more reperfusion information is acquired at other times to increase temporal resolution of the perfusion.

In act 48, the contrast agents are detected. The detection occurs in response to the scanning of act 46. Any contrast agent detection may be used. Contrast agents may be detected in response to single pulses for each scan line, such as using B-mode or intensity detection. Filtering to better isolate response at the second or higher harmonics or fractional harmonics may be used.

In preferred embodiments, the detection relies on combinations of signals from a plurality of transmit pulses. The received echoes from each transmission are combined to detect contrast agent response. For example, three or more receive signals representing a same location are combined to determine the nonlinear fundamental response at the location. The corresponding transmit pulses have different phases and amplitudes. Nonlinear fundamental response is greater for contrast agents than tissue, so provides good specificity. Phase inversion using two transmit pulses with opposite phase and combining the received signals may be used. Other contrast agent detection may be used. In addition or alternative to different phases and/or amplitudes for transmitted pulses, different weights of the receive signals may be used. The reperfusion is detected using combinations of multiple pulses. The contrast agents in the tissue of the region of interest at a given scan time are detected, indicating reperfusion at that time past the destruction or other introduction of contrast agents to the region.

Use of multiple pulses may be slower than single pulse detection. For example, using three transmit pulses with different phases and/or amplitudes and combining the responsive echo signals scans three times slower than single pulse B-mode detection for a same spatial resolution and scan size. Two pulse-based techniques are twice as slow as corresponding single pulse B-mode or filtered harmonic B-mode techniques.

The receive signals from a multiple pulse contrast agent detection may be used to generate other information, such as using one of the sequence of multiple pulses for B-mode imaging. This information may be used to supplement or assist in tracking, such as providing a separate motion measurements using the sub-volume for combination with (e.g., average) and/or verification of (e.g., within a threshold amount of difference) of the tracking of act 30.

The scanning and detection of acts 46 and 48 are performed at a greater spatial resolution than the scanning and reception of acts 32 and 34. Since acts 46 and 48 deal with the reperfusion to be measured, a greater spatial and/or temporal resolution may be desired. The tracking may be performed with a lesser resolution, increasing the overall scan rate. In alternative embodiments, the perfusion detection is performed at a lesser or the same resolution as the reception for tracking.

The detection of reperfusion acts 44-48 are performed once, providing an indication of reperfusion from destruction to the time of scanning for contrast agents. In a preferred embodiment, the detection acts 44-48 are repeated. The scanning and detection of acts 46 and 48 are repeated a plurality of times during a given physiological cycle, such as 20, 30, 40 or other number of times per cycle for a portion of a cycle, one cycle, or a plurality of cycles.

The repetition is with or without interleaving of the scanning for tracking. The scans for contrast agent detection may be used to replace or verify predicted tracking without interleaving separate scanning for tracking. The motion may be predicted based on previous cycles without tracking in a given cycle. Even with interleaving, scanning for detection of perfusion may be performed 20 or more times a second or per cycle, but may be performed less. A rate of 30 or more scans a second provides a high temporal resolution. Since the sub-volume rather than the entire volume is being scanned with multi-pulse detection of contrast agents, the scanning may be performed more rapidly than scanning the entire volume.

For each repetition, the scan line positions and depth are altered based on the predicted location of the tissue of interest. As the tissue moves, the beamformation parameters are altered to scan the region of interest. Since movement at the time of scanning is not known until after scanning, the motion is predicted based on past motion at the same or similar phase of the cycle or a current trend.

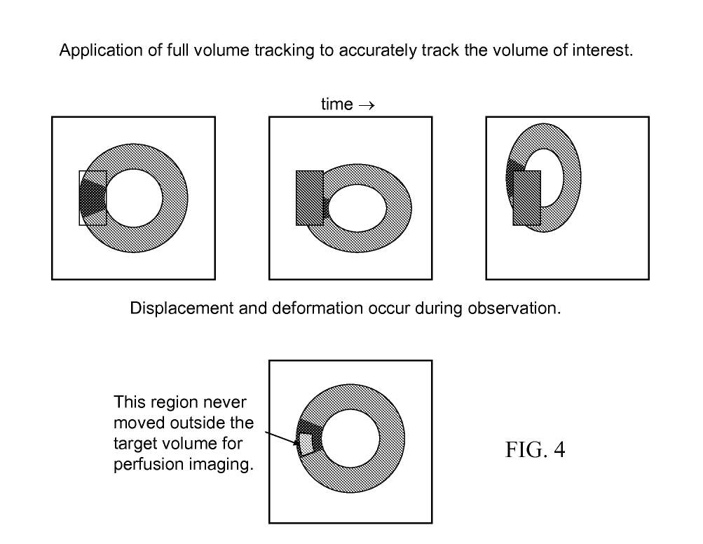

FIG. 4 shows one example of performing acts 30, 40, and 42 in two dimensions for simplicity. Tracking is shown in azimuth and elevation. However, tracking is also performed in the sound propagation direction (depth or range) or three-dimensions.

The region of interest is the wedge or sector within the annulus representing the heart walls. The target perfusion imaging sub-volume is shown as the rectangular box. As the organ moves and deforms, the sub-volume and/or the region of interest is tracked. The beamforming parameters are not changed. The sub-volume is static in position, but large enough to cover at least part of the tissue of interest throughout the cycle of motion. The tracking is used to determine which portion of the tissue has perfusion data for what portions of the cycle. For example, one small region of tissue may have been included within each repetition of the scan.

For the small region, the temporal resolution for the perfusion determination is greatest. Other regions may have less temporal resolution since the tissue is beyond the scan field for one or more repetitions. The tracking allows indication of the higher temporal resolution data.

Alternatively, the perfusion map is computed only for that part of the tissue of interest that did not move out of the scan sub-volume. This provides for perfusion quantification from only the small region. The tracking allows perfusion quantification less affected by tissue motion. The data representing perfusion at a given location is determined by accounting for motion through tracking. For a given spatial location in the small region, the same tissue is used for each time increment in the reperfusion. Tracking allows removal or adjustment for the motion.

The volume bounded by the outer box is scanned for tracking. The sub-volume or smaller box is scanned for contrast agent destruction and detection. The use of contrast specific pulse sequences for the sub-volume ensures that a high frame rate acquisition can be achieved.

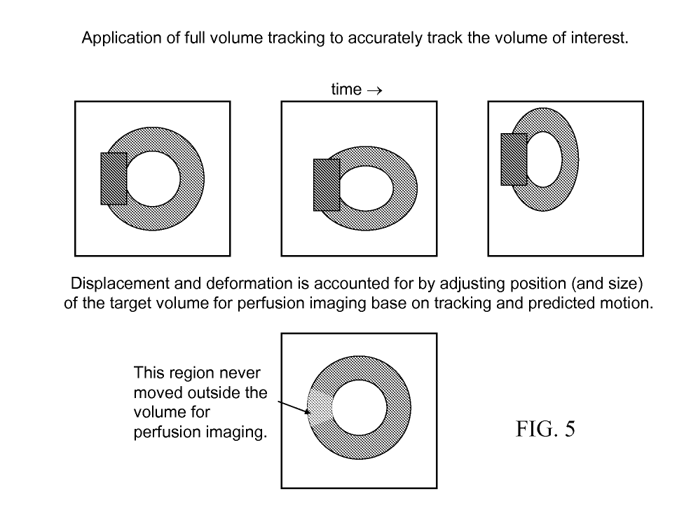

FIG. 5 shows another example embodiment, represented in two-dimensions but used in three-dimensions. In this example, the tracking is used to steer the beams. The information from tracking using the scan of the full volume is used in adjusting the target perfusion volume or sub-volume. The sub-volume scan box shifts so that all or more of tissue of interest is contained within the sub-volume while moving through the regular cycle. The displacement, scale, and/or deformation of the tissue volume of interest are accounted for by adjusting the position, size, and/or shape of the target perfusion volume or sub-volume. The ability to track the motion ensures that the contrast specific pulse sequences are applied only for that sub-volume, and a higher frame rate can be achieved. The high frame rate, high resolution sub-volume contrast specific image may be used for generating perfusion maps of the tissue region of interest. Unlike the example of FIG. 4, the perfusion map with the highest temporal resolution is created for the entire tissue volume of interest (e.g., the wedge region).

Referring again to FIG. 1, an image of the reperfusion is generated in act 50. An image of the tissue of interest at one time after destruction indicates an amount of perfusion for each location over the time period. A sequence of images may indicate the change in perfusion over time.

In one embodiment, an image is generated of the perfusion over time. Using the tracking, the amount of perfusion for a given location relative to the tissue may be determined. For a given volume location or voxel, the difference in contrast agent response between two times may be determined. The determination may be performed from an assumed zero response immediately after destruction without the tracking to align spatial locations. The tracking to align the scanning provides the data at the appropriate locations. For determining rate between times other than the destruction time, the tracking may be used to align the tissue locations before taking the difference in amount of contrast agent response.

The difference in amount of contrast agent response divided by the time separating the scans for the data indicates a rate. The rate may be used to modulate a display characteristic, such as color or intensity. The image represents the perfusion rate. The rate is determined for each location in the tissue of interest. The modulation may alternatively be a function of the difference in contrast agent response or the contrast agent response without further quantification. Other perfusion values may be used.

In one embodiment, the image is an unwrapped representation of the tissue of interest. For example, the tissue of interest is part of the heart wall. This three-dimensional structure is unwrapped to provide a two-dimensional representation, such as flattening a globe. The perfusion rate is mapped to the pixels of the unwrapped representation. The perfusion for a given pixel may be determined by the perfusion at a given depth, such as the median, of the heart wall or a combination (e.g., averaging along the thickness of the heart wall).

In other embodiments, a three-dimensional rendering is generated from the perfusion data. Any rendering may be used, such as volume, projection (maximum, minimum, alpha blending), or surface rendering. The contrast agent response and/or the calculated perfusion rate at the different locations in the volume may be used for the rendering values or voxels. The image may be re-rendered for different times, viewing directions, and/or rendering settings. Two-dimensional images, such as a multiplanar reconstruction, may be generated from the perfusion information representing the sub-volume.

In one embodiment, the perfusion information is included with an image of the tissue. A three-dimensional rendering from the full volume scan is generated in act 52, such as a three-dimensional B-mode rendering. The perfusion information is overlaid with or also used for rendering, such as rendering the tissue information with more transparency and/or as grayscale and rendering the perfusion information as more opaque and/or color. Alternatively, separate tissue and perfusion (contrast agent) images are rendered and displayed adjacent to each other.

In an example rendering, a sequence of three-dimensional representations is generated from the volumes. The volumes and sub-volumes are interpolated to a three-dimensional grid. Alternatively, the rendering is performed from data in an acquisition or other format. Ray casting or other rendering techniques may be used. The data may be classified or transformed, such as for opacity or color. More than one type of data may be used, such as having different or combined volumes for different types of ultrasound data (e.g., B-mode and perfusion data).

By displaying the sequence of three-dimensional representations, the change in shape, size, and/or position of a cyclic organ may be shown using the volume scan information. The change in perfusion over time may be shown by rendering a series of perfusion images. The change is shown over one or more cycles, but may be for only a portion of a cycle. One or more volumes associated with a specific phase, such end diastole or systole, may be used to generate specific three-dimensional representations without displaying the sequence. By aligning the frames of data spatially and temporally, one or more desired volumes are available for imaging or analysis.

The sub-volume may be increased in size, but at a reduction in temporal resolution. As an alternative, acts 30, 40, and 42 are repeated for different sub-volumes. All of the tracking, destruction and perfusion detection acts may be repeated for different tissue regions. Perfusion information is acquired with the desired temporal resolution for each region sequentially.

In one embodiment, the user indicates all of the tissue for which perfusion information is sought. Since the system tracks of the motion of the organ, the acquisition can be automated. Depending on the size of the tissue relative to the volume and a desired temporal resolution, the tissue may be divided into smaller sub-volumes. The division may occur automatically or manually. The system sub-divides the structure into one or more sub-volumes to allow enough temporal resolution and accurate tracking for each of the sub-volumes. Destruction/reperfusion imaging is done sequentially for these sub-volumes.

The individual results from each of the plurality of sub-volumes are reassembled and displayed in a same image, but may be displayed separately. Perfusion maps for several tissue volumes of interest may be computed. Alternatively, tissue volumes of interest covering the entire organ are selected automatically and perfusion maps are computed for the entire organ. The image represents the perfusion over time for all or a sub-set of the multiple sub-volumes. These sub-volumes can be spatially matched with the anatomy based on tracking information and registration. A complete perfusion profile may be constructed for the entire three-dimensional volume by combining several sub-volumes. Alternatively, these sub-volumes are individually studied with appropriate spatial matching with the complete three-dimensional volume.

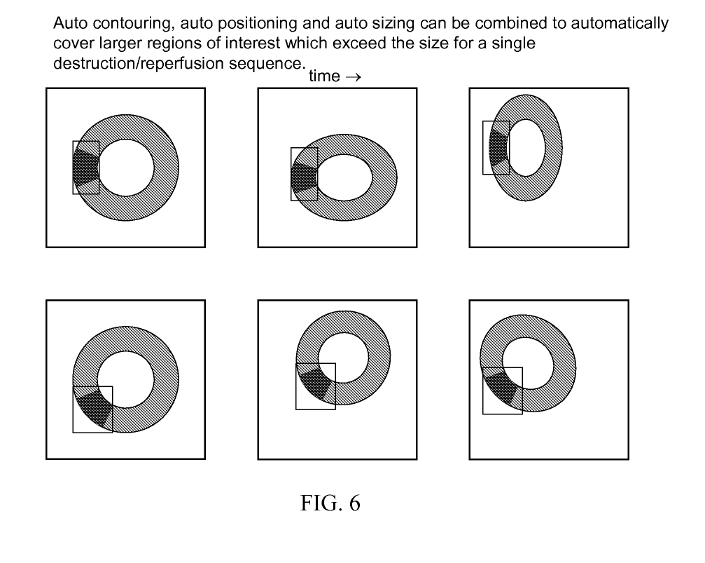

FIG. 6 shows one example of use of two sub-volumes. Additional sub-volumes may be systematically defined to cover the entire organ. The user or the system automatically selects or creates sub-volumes for perfusion quantification. In FIG. 6, the two rows exemplify the sequential perfusion assessment for two neighboring sub-volumes. The same or different scans are used to determine the motion history for each sub-volume. Different, sequential scans are used for destruction and reperfusion detection for each of the sub-volumes.

FIG. 7 shows a system 10 for perfusion imaging of a volume in medical diagnostic ultrasound. In one embodiment, the system 10 is a medical diagnostic ultrasound system. The system 10 is a system for scanning, a workstation or personal computer. The system 10 includes a transducer 12, a beamformer 14, a processor 16, a detector 18, a contrast agent detector 20, a memory 22, and a display 24. Additional, different, or fewer components may be provided. For example, the system 10 does not include the transducer 12, beamformer 14, detector 18, and/or contrast agent detector 20. Instead, the system 10 is a computer or workstation that receives detected ultrasound data from an imaging system. In other embodiments, the system 10 is a different type of medical system and associated components, such as magnetic resonance, positron emission, computed tomography or x-ray imaging system. The system 10 is used for scanning any cyclically moving object, such as the heart, lungs, stomach, diaphragm, or vessels. The system 10 implements the method of FIG. 1 or a different method.

The transducer 12 is an array of elements, such as piezoelectric or capacitive elements. The array is a one-dimensional or multi-dimensional distribution of elements. For example, the transducer 12 is a two-dimensional array for scanning a volume electronically. One possible multi-dimensional transducer array is a matrix probe (e.g., a 4Zlc probe from Siemens Medical Solutions USA, Inc.) As another example, the transducer 12 is a wobbler transducer array for scanning in one dimension electronically and in another dimension mechanically. Other now known or later developed transducers 12 for mechanical and/or electrical steering of different planes may be provided. For example, a user may move a one dimensional array transducer manually or robotically to a new location for each plane.

The beamformer 14 is a transmit beamformer, receive beamformer, combinations thereof, or other now known or later developed device for scanning a region with the transducer 12. In one embodiment, the beamformer 14 includes transmitters or waveform generators for generating electrical waveforms for each element of a transmit aperture. The waveforms are associated with phase and amplitude. The waveforms for a given transmit event may have the same or different phasing. The electrical waveforms are relatively weighted and delayed to form an acoustic beam with a desired phase and amplitude characteristic. For example, the transmit beamformer includes amplifiers, phase rotators, and/or controllers to generate sequential, steered pulses with the desired phase and amplitude in relation to other acoustic beams. Converging, diverging or planar beams may be used.

The beamformer 14 may include receive beamformers, such as delays, phase rotators, amplifiers, and/or adders for relatively delaying and summing received signals to form one or more receive beams with dynamic focusing. For example, using shared processing, separate processing, or combinations thereof, a plurality (e.g., tens or hundreds) of parallel receive beamformers are provided to form a respective plurality of receive beams in response to a given transmit beam. Alternatively, the beamformer 14 includes a processor for Fourier or other analysis of received signals to generate samples representing different spatial locations of the scanned region.

The transducer 12 and beamformer 14 are configured to scan a volume and a sub-volume. The beamformer is controlled or programmed to perform the scan. The beamformer parameters, such as relative delays and/or phasing for focus, apodization, beam amplitude, beam phase, frequency, or others, are set. The aperture for transmit and the aperture for receive on the transducer 12 is set. The beamformer 14 and transducer 12 are used to generate the waveforms for the aperture and convert the waveforms to acoustic energy for transmitting the beam, and used to receive acoustic energy at the receive aperture, convert the acoustic energy to electrical energy, and beamform the received electrical signals.

Electric and/or mechanical steering may be used to scan the volume and/or sub-volume. A volume scan may be performed using any pattern or distribution of scan lines. In one embodiment, an acquisition scan plane is positioned within a three-dimensional region. Acoustic energy is transmitted in any of various now known or later developed scan patterns along the scan plane for acquiring data. The scan plane is then altered to another location in the volume or sub-volume and scanned.

For a given volume or sub-volume, the scans may be repeated. By repeating the scans, a sequence of frames of voxel data is obtained. Each frame represents the entire three-dimensional scanned volume or sub-volume, but may only represent smaller regions within the volume or sub-volume. By repeating the scanning, a plurality of frames of beamformed data representing the volume and/or sub-volume within a given cycle is acquired. The scans of the volume may be interleaved with scans of the sub-volume. Any of scan line, part of frame, frame, or group of frame interleaving may be used. For example, initially, the volume is scanned without scanning of the sub-volume. After one or more physiological cycles, a destruction scan for the sub-volume is interleaved with the volume scanning. Reperfusion detection scanning of the sub-volume is then interleaved with the volume scanning, such as performing one, two, or more scans of the sub-volume interleaved with each scan of the volume.

The volume scanned for tracking is larger than the sub-volume being tracked. This allows for determination of the shift in the tissue of interest and/or motion tracking for multiple tissue regions. In alternative embodiments, the volume used for tracking is the same size as the sub-volume used for destruction and/or reperfusion scanning.

The beamformer 14 is configured to scan the sub-volume or tissue of interest based on the tracked motion. The scan for destruction and/or reperfusion is performed as a function of the tracking. The tracking is used to determine the location (e.g., steering) and extent of the scan pattern (e.g., depth and lateral extent/number of scan lines) for scanning the sub-volume.

By adjusting the beamforming, the contrast agents in the region of tissue are destroyed and contrast agents outside of the scan region or sub-volume are less likely to be destroyed. Contrast agents are more likely destroyed by higher amplitude, greater scan line density, greater pulse repetition, lower frequency, and/or other parameter for increasing the destructive power. By placing the focal region within the sub-volume, contrast agents within the sub-volume are more likely destroyed. Contrast agents spaced laterally from the sub-volume are less likely destroyed based on the roll-off of the acoustic amplitude or distance away from the transmit beams. Due to phased array focusing, contrast agents at deeper and/or shallower depths may be subjected to less destruction. The focal point may be varied to more thoroughly destroy contrast agents in the tissue of interest and/or to account for the effects of the contrast agents on the focus.

By adjusting the beamforming, the contrast agents in the region of tissue may be detected for reperfusion quantification. The tracking is used to predict a location of the tissue or sub-volume during the scanning. The location is updated throughout the physiological cycle so that the sub-volume includes the moving region of tissue at each of different scan times. The sub-volume scan changes position, shape, and/or size in correspondence with the tracked position, shape, and/or size of the region of tissue.

The detector 18 is an ultrasound detector. The detector is configured by hardware and/or software to detect from the beamformed data. Any detection may be used, such as B-mode, Doppler or color flow mode, harmonic mode, or other now known or later developed modes. B-mode and some harmonic modes use single pulse scan techniques for detection. The intensity of the received signals in the frequency band of interest is calculated. Multiple pulse techniques, such as flow mode estimation of velocity or energy, may be used.

The detector 18 detects the response to the transmit beams for the scan of the volume. The spatial and/or temporal resolution of the detected data is based on the beamforming or scanning resolution. Detected data representing the volume is provided. Such frames of data are provided for the same or similar volumes (e.g., similar accounts for unintended transducer and/or patient movement offsetting the volume) at different times throughout the cycle.

The processor 16 is configured to determine and/or receive an indication of the region of interest and to track the region of interest. The configuration is provided by hardware and/or software. The processor 16 is a general processor, control processor, application-specific integrated circuit, field-programmable gate array, digital circuit, analog circuit, digital signal processor, combinations thereof, or other now known or later developed device for spatial and temporal alignment of acquired data. The processor 16 is a single device or group of devices. For example, the processor 16 includes separate processors operating in parallel or sequence. As another example, the processor 16 includes a network of devices for distributed processing in parallel or sequence. In one embodiment, the processor 16 includes a three-dimensional image rendering processor, such as a graphics processing unit, graphics card, or other device for rendering.

The processor 12 tracks the moving region of tissue. The tracking determines the similarity of a reference sub-volume of data with a later volume. Different translations, rotation, scales, and/or deformations are searched and one with a highest or sufficient similarity is selected. The process is repeated for subsequent volumes through a cycle. For example, matching is used to determine the position and size of the region of tissue as a function of time throughout one or more cycles. The location and size of the tissue at different times within the cycle is determined. The tracking information may be used to control the beamformer 14 or used by the beamformer 14.

The contrast agent detector 20 is configured to detect response from contrast agents. The configuration is provided by software and/or hardware. The contrast agent detector 20 is a B-mode detector, Doppler or flow estimator, contrast agents specific detector or other now known or later developed device for detecting acoustic response of contrast agents. The contrast agent detector 20 may include a filter, summer, memory, buffer, rectifier, or other components for combining data responsive to different transmissions. The contrast agent detector 20 is a different device from the detector 18, but may be a same device used sequentially with the same or different settings.

The detected response may include other information, such as second harmonic, even harmonic, B-mode, velocity or power estimates including information from tissue, moving tissue, and/or blood. Alternatively, the detected response is specific to contrast agents, such as using a combination of receive signals responsive to transmit pulses with different phase and amplitude to detect contrast agent while limiting response from tissue. For example, the nonlinear fundamental response is detected by combining three or more receive signals responsive to transmit beams with different phasing and amplitude.

The beamformed data from the scans of the sub-volume or tissue volume of interest is provided to the contrast agent detector. The response from at least contrast agents in the sub-volume is detected. Since the scan region varies with movement of the tissue, the detected contrast agent response for different times is for the same tissue.

The processor 12 operates on any ultrasound data for tracking, such as data output by the beamformer 14, detector 18, contrast agent detector 20, or at other points along the ultrasound data path. The ultrasound data corresponds to beamformed, detected (e.g., B-mode, velocity, energy, power, variance, harmonic, contrast agent, or combinations thereof), and/or scan converted data.

The processor 12 or a separate processor generates images from the volume scan and/or sub-volume scan. For example, grayscale and/or color coding is used to generate a perfusion map from tissue and contrast agent data. The processor 12 may render images from voxels. Any image is output to the display 24.

The display 24 is a CRT, LCD, plasma, projector, printer, or other now known or later display device. The display 24 receives the image data from the processor 12 or other component and generates the image. A perfusion map, three-dimensional rendering, two-dimensional image, or other image is displayed. For example, a perfusion map is generated as a function of the detected contrast agents, such as modulating pixels by the perfusion rate for locations representing the tissue. The perfusion map is displayed.

The memory 22 is a cache, buffer, RAM, removable media, hard drive or other computer-readable storage media. Computer-readable storage media include various types of volatile and non-volatile storage media. The functions, acts or tasks illustrated in the figures or described herein are performed by the processor 16 executing instructions stored in or on the computer-readable storage media of the memory 18. The functions, acts or tasks are independent of the particular type of instructions set, storage media, processor or processing strategy and may be performed by software, hardware, integrated circuits, firmware, microcode and the like, operating alone or in combination. Likewise, processing strategies may include multi-processing, multi-tasking, parallel processing and the like. In one embodiment, the instructions are stored on a removable media device for reading by a medical diagnostic imaging system. The imaging system uploads the instructions for performing the acts discussed herein. In another embodiment, the instructions are stored in a remote location for transfer through a computer network or over telephone lines to an imaging system or workstation. In yet other embodiments, the instructions are stored within the imaging system or workstation.

The memory 22 alternatively or additionally stores the collected ultrasound data and/or spatial or temporal information.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (We) claim:

1. A method for perfusion imaging of a volume in medical diagnostic ultrasound, the method comprising:
first scanning a volume of a patient with single pulses for each transmit scan line;
receiving first ultrasound data in response to the single pulses;
repeating the first scanning and receiving a plurality of times during a first physiological cycle;
determining a region of interest of the patient within the volume from the first ultrasound data of at least one of the first scans;
tracking the region of interest between the repeated first scans with the first ultrasound data from the repeated first scans;
predicting a first next location of the region of interest as a function of the tracking;
second scanning a sub-volume of the volume, the sub-volume corresponding to the region of interest at the first next location, beamforming parameters being set for the first next location to perform the second scanning of the sub-volume and not laterally outside the sub-volume, the second scanning comprising acoustic energy for destroying contrast agents;
predicting a second next location of the region of interest as a function of the tracking;
third scanning the sub-volume, the beamforming parameters being set for the second next location for the third scan of the sub-volume, the third scanning comprising acoustic energy for detecting the contrast agents;
detecting the contrast agents in response to multiple pulses from the third scanning;
repeating the third scanning and detecting a plurality of times during a second physiological cycle; and
generating an image representing perfusion over time of the contrast agents in the sub-volume, the image responsive to the detected contrast agents of the repetitions of the third scanning.

2. The method of claim 1 wherein the third scanning and detecting comprise detection of non-linear fundamental response of the contrast agents using combinations of response from three or more pulses of the third scanning.

3. The method of claim 1 wherein the repeating of the first scanning and receiving continues to occur interleaved with the repeating of the third scanning and detecting.

4. The method of claim 1 wherein the predicting comprises predicting the first and second next locations from previous motion from, at least in part, the first physiological cycle at a similar phase of the physiological cycle.

5. The method of claim 1 wherein the first, second and third scanning occur in combination at a rate at least greater than 20 Hz.

6. The method of claim 1 wherein generating the image comprises mapping a perfusion rate to pixel locations representing the region of interest.

7. The method of claim 1 further comprising generating a three-dimensional rendering with the first ultrasound data, the image being included on a display with the three-dimensional rendering.

8. The method of claim 1 wherein the receiving of the first ultrasound data is performed at a lower spatial resolution than the detecting of the contrast agents.

9. The method of claim 1 further comprising repeating all of the acts for a different sub-volume, wherein the image represents the perfusion over time for the sub-volume and the different sub-volume.

10. The method of claim 1 wherein the third scanning comprises scanning with a mechanical index for maintaining contrast agents without substantial destruction.

11. A system for perfusion imaging of a volume in medical diagnostic ultrasound, the system comprising:
a transducer and beamformer configured to scan a volume and a sub-volume, the volume larger than the sub-volume, a first scan of the sub-volume configured to destroy contrast agents in the sub-volume;
a detector configured to detect response to the scan of the volume;
a processor configured to track a moving region of tissue as a function of time within the volume using the response to the scan of the volume;
wherein the beamformer is configured, as a function of the tracking, to perform second scans of the sub-volume at different locations at different times such that the sub-volume includes the moving region of tissue;
a contrast agent detector configured to detect contrast agents in response to the second scans of the sub-volume; and
a display configured to display an image, the image being a function of the detected contrast agents.

12. The system of claim 11 wherein the contrast agent detector is configured to detect the contrast agents in the sub-volume at different times, the image representing a perfusion rate of the contrast agents in the region of tissue.

13. The system of claim 11 wherein the beamformer is configured to perform the first scan as a function of the tracking such that the scan destroys contrast agents in the region of tissue and is less destructive at regions laterally spaced from the region of tissue, the region of tissue being a three-dimensional region.

14. The system of claim 11 wherein the processor is configured to track position and size of the region of tissue as a function of time, and wherein the beamformer is configured to scan the sub-volume where the sub-volume changes position and size in correspondence with the tracked position and size of the region of tissue.

15. The system of claim 11 wherein the detector comprises a B-mode detector configured to detect the response using single pulses, and wherein the contrast agent detector is configured to detect the contrast agents using combinations of pulses.

16. In a computer readable storage medium having stored therein data representing instructions executable by a programmed processor for perfusion imaging of a volume in medical diagnostic ultrasound, the storage medium comprising instructions for:
tracking tissue motion in three-dimensions of a first volume;
adjusting, as a function of the tracking, beam steering to account for the tissue motion, the adjusting being ongoing throughout a physiological cycle;
transmitting acoustic energy destructive of contrast agents with the adjusted beam steering; and
detecting, after the transmitting of the acoustic energy destructive of the contrasts agents, reperfusion in a second volume over time, the detecting performed with the adjusted beam steering, the second volume being a same volume as the first volume or being based on the tracking of the first volume.

17. The computer readable storage medium of claim 16 wherein the tracking is performed in response to B-mode scanning and detection, and wherein the detecting comprises detecting the reperfusion using combinations of multiple pulses.

18. The computer readable storage medium of claim 17 wherein the combinations of multiple pulses comprises combinations resulting in the detecting of nonlinear fundamental response of the contrast agents.

19. The computer readable storage medium of claim 16 wherein the tracking is performed with scanning of a third volume larger than the first volume.

20. The computer readable storage medium of claim 16 further comprising generating an image of the reperfusion.
